(19) United States
(12) Patent Application Publication
(10) Pub. No.: US 2022/0010965 A1
(43) Pub. Date: Jan. 13, 2022

(54) INTELLIGENT ELECTRIC FIREPLACE

(71) Applicant: Dong Guan Song Wei Electric Technology Co., Ltd., Dongguan City (CN)

(72) Inventor: Weilin Lu, Dongguan City (CN)

(21) Appl. No.: 16/923,094

(22) Filed: Jul. 8, 2020

(51) Int. Cl. F24C 7/00; F24C 7/08; F24F 11/30; A61L 9/20

(52) U.S. Cl. CPC F24C 7/004; F24C 7/081; F24F 2110/50; A61L 9/20; F24F 11/30

(57) ABSTRACT

An intelligent electric fireplace includes a housing, a simulated charcoal and a first light-emitting device. The first light-emitting device is configured to emit light toward the simulated charcoal to form a burning effect of the simulated charcoal. An ultraviolet germicidal lamp is turned on/off according to an air quality condition fed back by an air quality sensing module to reduce a waste caused by the ultraviolet germicidal lamp that is still used when the air quality condition meets the standard. It is more energy-saving and environmentally friendly, and it is more intelligent.

### BACKGROUND OF THE INVENTION

### 1. Field of the Invention

The present invention relates to an electric fireplace, and more particularly to an intelligent electric fireplace.

### 2. Description of the Prior Art

In modern society, electric fireplaces are developed rapidly instead of traditional fireplaces. While people enjoy the convenience brought by electric fireplaces, more modern needs are required. For example, traditional electric fireplaces usually only have the function of heating, providing a single function. Environmentally friendly electric fireplaces are developed accordingly, which use ultraviolet germicidal lamps to disinfect the air before heating, and the structure is relatively complicated. Moreover, the ultraviolet germicidal lamp is used all the time, which consumes a lot of electricity, and the ultraviolet germicidal lamp needs to be replaced frequently.

Accordingly, the inventor of the present invention has devoted himself based on his many years of practical experiences to solve this problem.

### SUMMARY OF THE INVENTION

In view of the shortcomings of the prior art, the primary object of the present invention is to provide an intelligent electric fireplace. By providing an air quality sensing module and an ultraviolet germicidal lamp in the electric fireplace, the ultraviolet germicidal lamp can be turned on/off according to the air quality condition fed back by the air quality sensing module to reduce a waste caused by the ultraviolet germicidal lamp that is still used when the air quality condition meets the standard. It is more energy-saving and environmentally friendly, and it is more intelligent.

In order to achieve the above object, the present invention adopts the following technical solutions:

An intelligent electric fireplace comprises a housing. A simulated charcoal and a first light-emitting device are provided in the housing. The first light-emitting device is configured to emit light toward the simulated charcoal to form a burning effect of the simulated charcoal.

The housing includes an air inlet, an air outlet, a heating device, a control device, an air quality sensing module, and an ultraviolet germicidal lamp. The first light-emitting device, the heating device, the air quality sensor module and the ultraviolet germicidal lamp are electrically connected to the control device, respectively. The ultraviolet germicidal lamp is turned on/off according to an air quality condition fed back by the air quality sensing module.

Compared with the prior art, the present invention has obvious advantages and beneficial effects. Specifically, it can be known from the above technical solutions:

1. By providing the air quality sensing module and the ultraviolet germicidal lamp in the electric fireplace, the ultraviolet germicidal lamp can be turned on/off according to the air quality condition fed back by the air quality sensing module to reduce a waste caused by the ultraviolet germicidal lamp that is still used when the air quality condition meets the standard. It is more energy-saving and environmentally friendly, and it is more intelligent.

2. It can realize automatic air quality detection, automatically turn on/off the ultraviolet germicidal lamp, and use the control device to immediately turn on the ultraviolet germicidal lamp for disinfection according to the air quality condition fed back by the air quality sensing module. When the air quality condition fed back by the air quality sensing module meets the standard, the control device controls the ultraviolet germicidal lamp to stop working. Preferably, the control device controls the light-emitting state of the first light-emitting device to be changed according to the air quality condition fed back by the air quality sensing module. The user can judge the current air quality condition intuitively through the burning state of the simulated charcoal, so the prompt effect is good.

3. The structural design of the whole electric fireplace is very clever.

The ultraviolet germicidal lamp is mounted to the underside of the middle plate. Through the accommodation space of the electric fireplace itself, the ambient air is in communication with the accommodation space through the meshed panel. The air flowing into the accommodation space is disinfected by the ultraviolet germicidal lamp. Generally, the front of the electric fireplace has a larger area, so the indoor air is in good communication with the accommodation space, and the ultraviolet germicidal lamp has a better disinfection effect.

The air quality sensing module is installed on the left, right or back of the upper space of the electric fireplace, keeping away from the air outlet of the electric fireplace, so that the detection of air quality is closer to the real situation of indoor air. Besides, it is more convenient to install the air quality sensing module on the left or right side of the upper space.

Furthermore, the middle plate extends obliquely upward from back to front, so that the light emitted by the ultraviolet germicidal lamp is irradiated outward toward the front side of the accommodation space, thereby further improving the disinfection effect of the ultraviolet germicidal lamp.

### BRIEF DESCRIPTION OF THE DRAWINGS

FIG. 1 is a perspective view according to a preferred embodiment of the present invention;

FIG. 2 is an exploded view according to the preferred embodiment of the present invention;

FIG. 3 is a partial enlarged view of FIG. 2;

FIG. 4 is a cross-sectional view according to the preferred embodiment of the present invention;

FIG. 5 is a front view according to the preferred embodiment of the present invention, not having the meshed panel;

FIG. 6 is a schematic view illustrating that the ultraviolet germicidal lamp is arranged horizontally;

FIG. 7 is a schematic view illustrating an installation structure of the ultraviolet germicidal lamp;

FIG. 8 is a schematic view illustrating that the ultraviolet germicidal lamp is arranged vertically;

FIG. 9 is a schematic view illustrating another installation structure of the ultraviolet germicidal lamp;

FIG. 10 is a front view according to the preferred embodiment of the present invention, having the meshed panel; and

FIG. 11 is a block diagram of an electric control connection according to the preferred embodiment of the present invention.

### DETAILED DESCRIPTION OF THE PREFERRED EMBODIMENTS

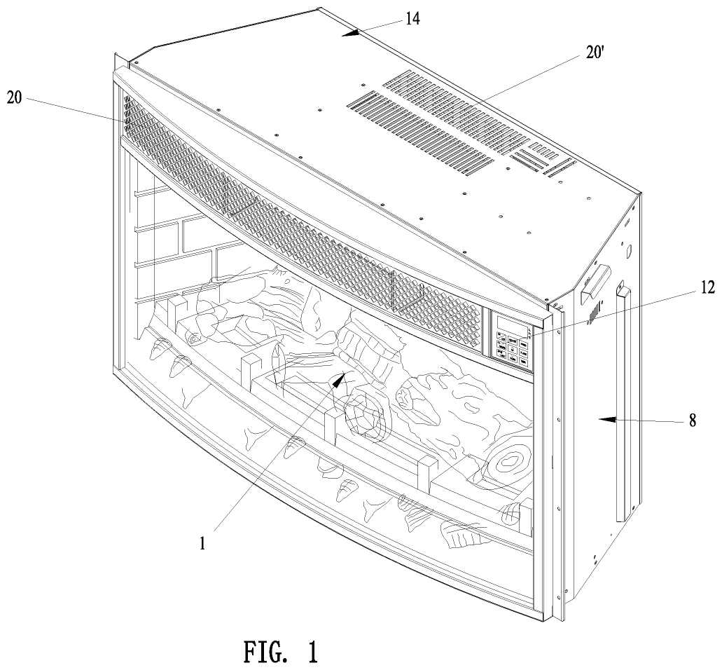

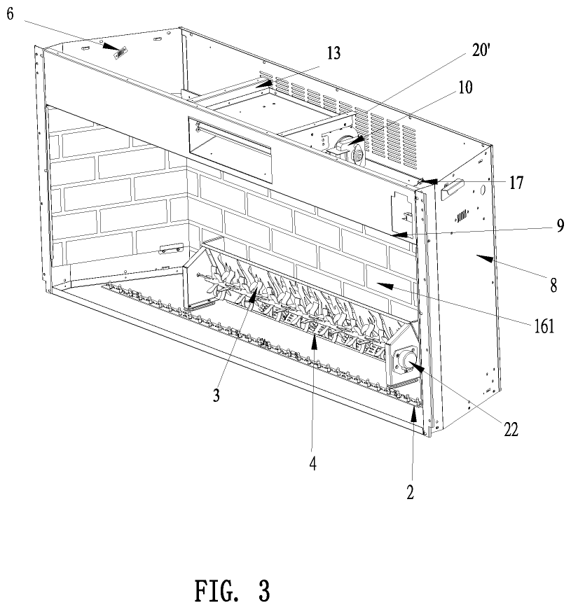

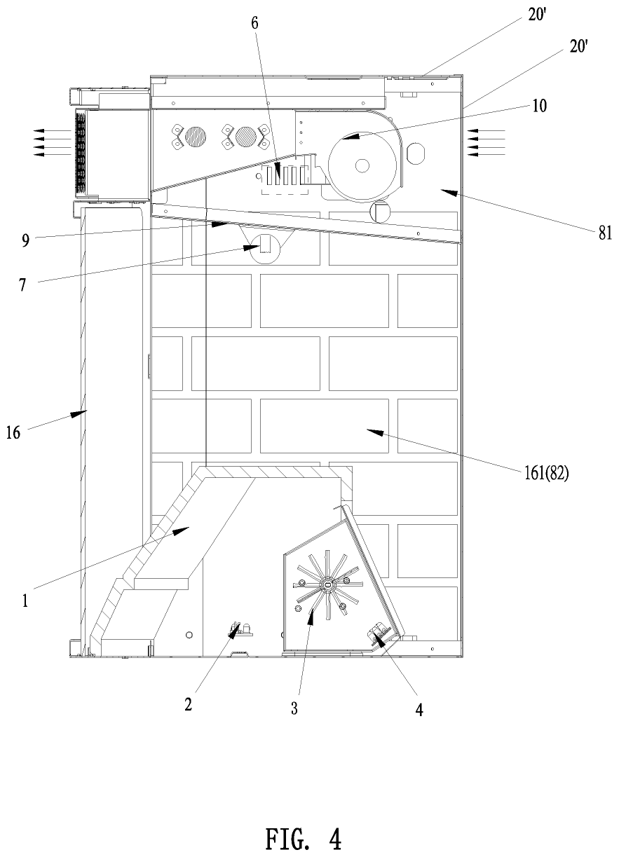

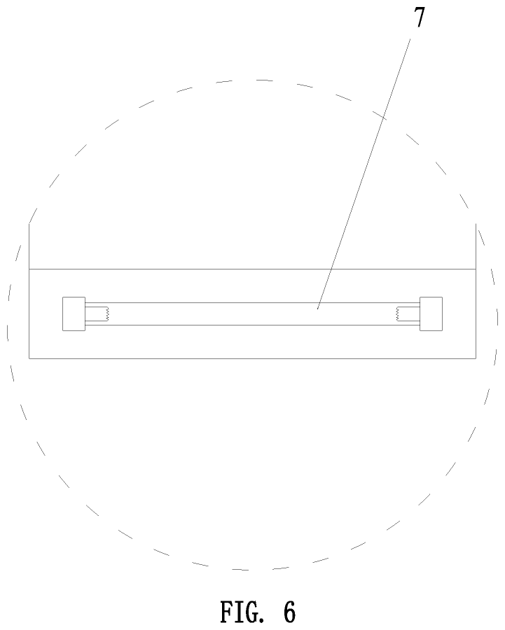

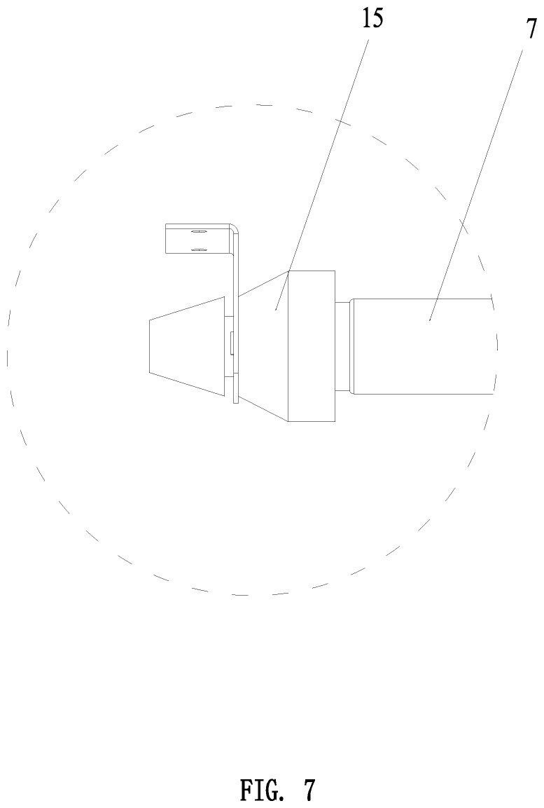

Referring to FIG. 1 through FIG. 11, there is shown a specific structure of an embodiment of the present invention.

An intelligent electric fireplace comprises a housing. A simulated charcoal 1 and a first light-emitting device 2 are provided in the housing. The first light-emitting device 2 is configured to emit light toward the simulated charcoal 1 to form a burning effect of the simulated charcoal 1. The first light-emitting device 2 is located below the simulated charcoal 1. A reflector 3 and a second light-emitting device 4 emitting light toward the reflector 3 are provided behind the first light-emitting device 2. The reflector 3 is equipped with a rotating motor 22 for controlling rotation of the reflector 3. The second light-emitting device 4 is mainly used for imaging the flame.

The housing includes an air inlet 20′, an air outlet 20, a heating device 21, a control device 5, an air quality sensing module 6, and an ultraviolet germicidal lamp 7. The first light-emitting device 2, the heating device 21, the air quality sensor module 6 and the ultraviolet germicidal lamp 7 are electrically connected to the control device 5, respectively. The ultraviolet germicidal lamp 7 is turned on/off according to the air quality condition fed back by the air quality sensing module 6.

The control device 5 controls the light-emitting state of the first light-emitting device 2 to be changed according to the air quality condition fed back by the air quality sensing module 6. The change of the light-emitting state means that the first light-emitting device 2 is in the first light-emitting state when the air quality condition fed back by the air quality sensing module 6 meets the standard; the first light-emitting device 2 is in the second light-emitting state when the air quality condition fed back by the air quality sensing module 6 does not meet the standard. The first light-emitting state and the second light-emitting state are visually different. For example, the first light-emitting state refers to static light emission, and the second light-emitting state refers to blinking light emission; or, the first light-emitting state and the second light-emitting state refer to light emission in different colors.

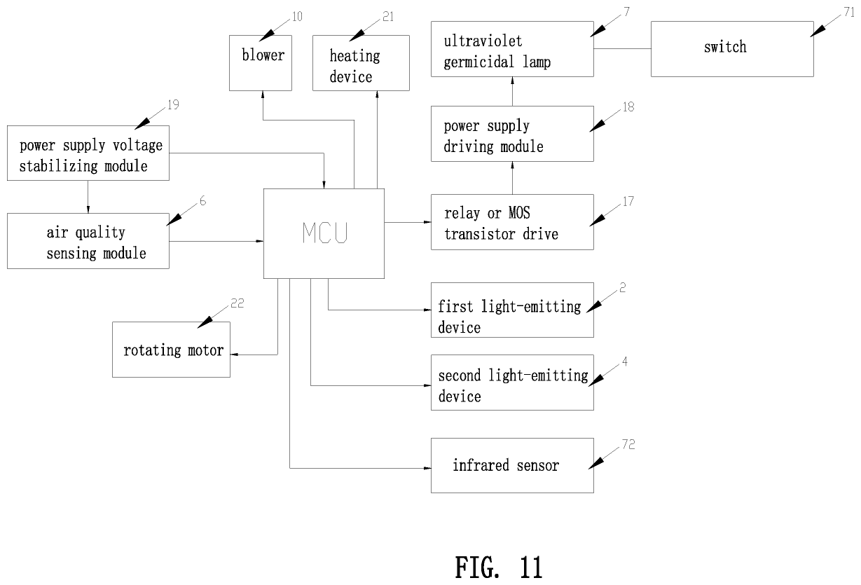

As shown in FIG. 11, the control device 5 includes an MCU (microprogrammed control unit). The air quality sensing module 6, the first light-emitting device 2 and the second light-emitting device 4 are electrically connected to the MCU, respectively. The MCU is connected to a power supply driving module 18 through a relay or MOS transistor drive 17, and further connected to the ultraviolet germicidal lamp 7. The power supply input end of the electric fireplace is connected to the air quality sensing module 6 and the MCU through a power supply voltage stabilizing module 19, respectively.

In this embodiment, by providing the air quality sensing module 6 and the ultraviolet germicidal lamp 7 in the electric fireplace, the control device turns on the ultraviolet germicidal lamp 7 for disinfection in a real-time manner according to the air quality condition fed back by the air quality sensing module 6. After the air quality condition fed back by the air quality sensing module 6 meets the standard, the control device controls the ultraviolet germicidal lamp 7 to stop working. This can effectively reduce the waste of energy when the air quality meets the standard and the ultraviolet germicidal lamp 7 is still in use.

The present invention may further comprise a switch 71 for manually controlling the ultraviolet germicidal lamp 7 to be turned on/off. When the light-emitting state of the first light-emitting device 2 shows that the air quality condition does not meet the standard, the user manually controls the switch 71 to turn on the ultraviolet germicidal lamp 7 for disinfection. Of course, both automatic control and manual control can be provided. For safety, the present invention may further comprise an infrared sensor 72 for detecting whether there is a person in front of the housing. The infrared sensor 72 is connected to the control device 5. When the infrared sensor 72 detects a person in front of the housing, the control device 5 controls the ultraviolet germicidal lamp 7 to stop working, so as to ensure personal safety.

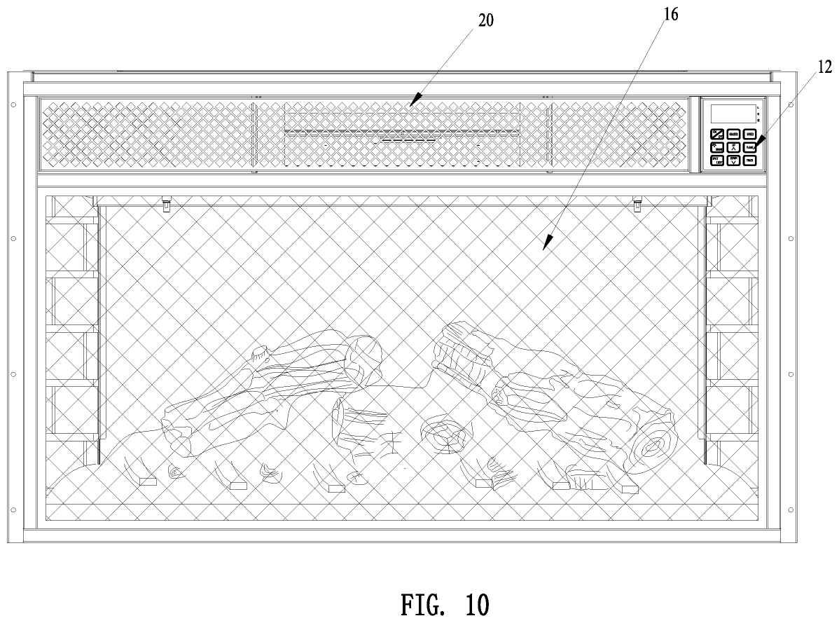

Preferably, a meshed panel 16 is provided on the front side of the housing, as shown in FIG. 10. An accommodation space 161 is formed between the inner wall of the housing and the meshed panel 16. The simulated charcoal 1 and the first light-emitting device 2 are located in the accommodation space 161. The ultraviolet germicidal lamp 7 is installed in the accommodation space 161. The ambient air is in communication with the accommodation space 161 through the meshed panel 16. The ultraviolet germicidal lamp 7 is configured to disinfect the air flowing into the accommodation space 161. On the one hand, the meshed panel 16 ensures sufficient air circulation. On the other hand, the meshed panel 16 also covers and protects the components in the accommodation space 161. Furthermore, the meshed panel 16 also plays a decorative role on the front side of the electric fireplace.

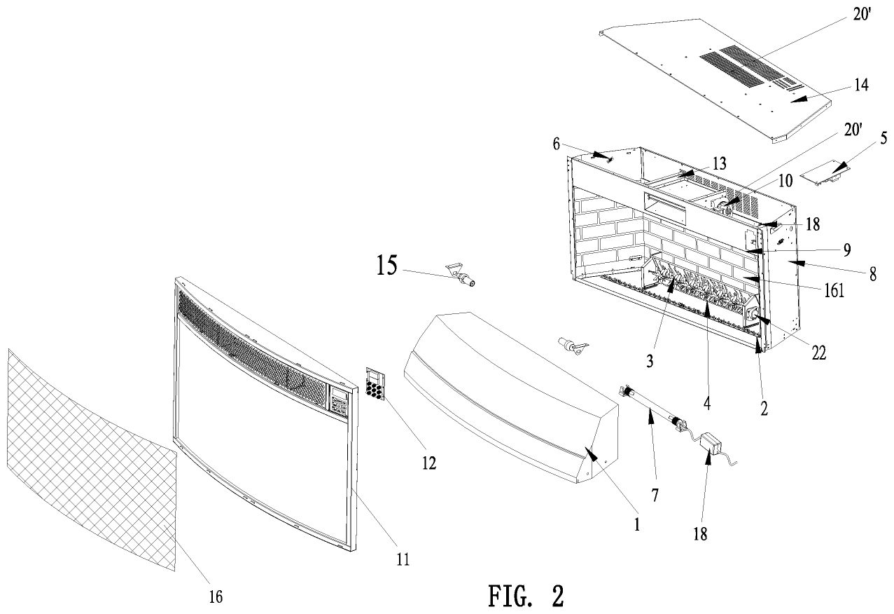

As shown in FIGS. 2 to 4, the housing includes a main housing 8 and a middle plate 9 disposed in the main housing 8. The middle plate 9 divides the main housing 8 into an upper space 81 and a lower space 82. The lower space 82 is defined as the accommodation space 161. The ultraviolet germicidal lamp 7 is installed to the underside of the middle plate 9. The middle plate 9 extends obliquely upward from back to front for providing an installation reference surface of the ultraviolet germicidal lamp 7 to extend obliquely upward from back to front, so that the light emitted from the ultraviolet germicidal lamp 7 is irradiated outward toward the front side of the accommodation space 161 to improve the disinfection effect of the ultraviolet germicidal lamp 7. A face frame 11 is provided on the front side of the main housing 8. The upper region of the face frame 11 is the air outlet 20. A button panel 12 is provided beside the air outlet 20. The middle and lower regions of the face frame 11 are configured to mount the meshed panel 16. The air inlet 20′ may be disposed on the rear side of the upper space 81 or a top cover 14. The air outlet 20 is disposed on the front side of the upper space 81. The heating device 21 is disposed in the upper space 81. A blower 10 is provided in the upper space 81 for forming an air flow from the air inlet 20′ to the air outlet 20. Generally, a blower bracket 13 is provided in the upper space 81 for installing and fixing the blower 10. The top cover 14 is installed on the top of the main housing 8 to cover the upper space 81.

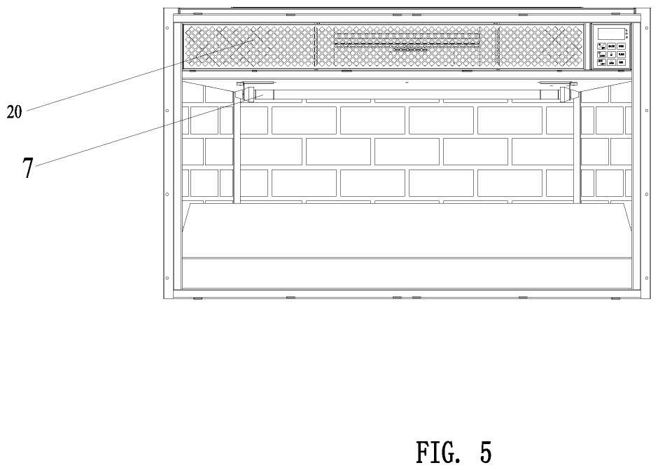

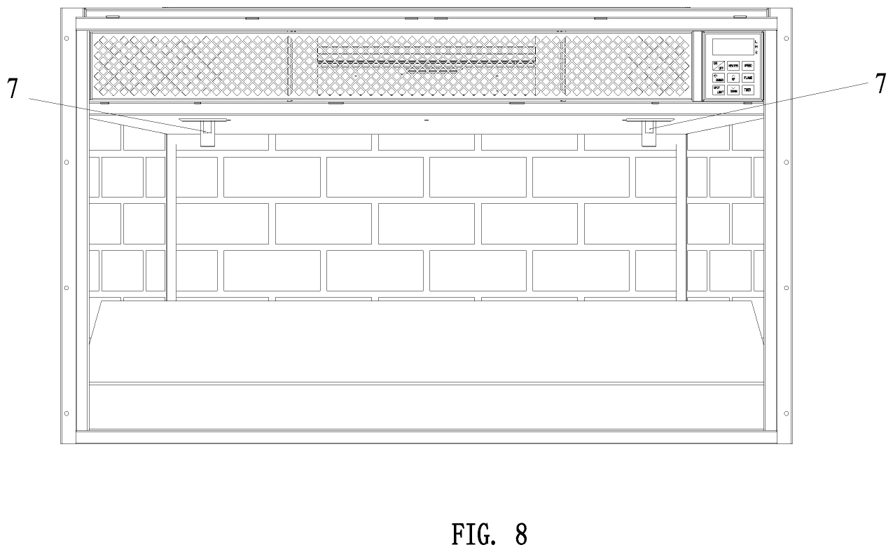

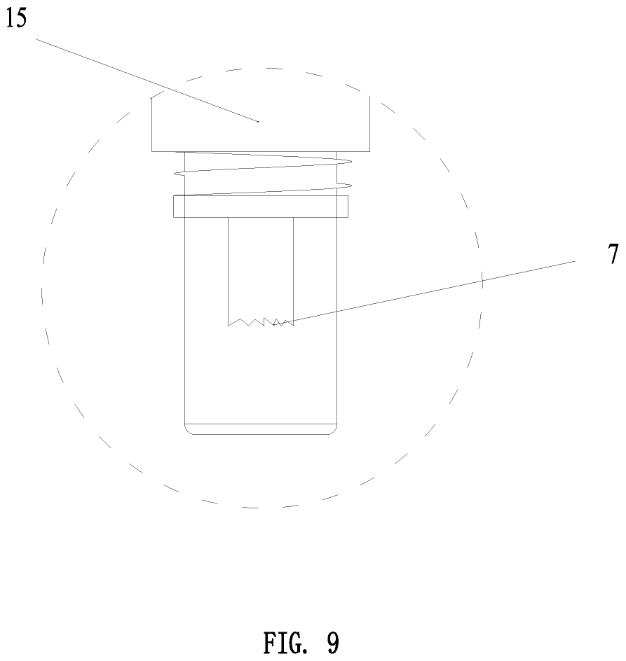

The air quality sensing module 6 is far away from the air outlet 20. Preferably, the air quality sensing module 6 is disposed on the left side or the right side or the rear side of the upper space 81. The ultraviolet germicidal lamp 7 is arranged horizontally or/and vertically on the underside of the middle plate 9. As shown in FIGS. 5 to 7, the ultraviolet germicidal lamp 7 is arranged horizontally. As shown in FIG. 8 and FIG. 9 the ultraviolet germicidal lamp 7 is arranged vertically. The ultraviolet germicidal lamp 7 is mounted and positioned by a lamp holder 15.

What is claimed is:

1. An intelligent electric fireplace, comprising a housing, a simulated charcoal and a first light-emitting device being provided in the housing, the first light-emitting device being configured to emit light toward the simulated charcoal to form a burning effect of the simulated charcoal; the housing including an air inlet, an air outlet, a heating device, a control device, an air quality sensing module, and an ultraviolet germicidal lamp; the first light-emitting device, the heating device, the air quality sensor module and the ultraviolet germicidal lamp being electrically connected to the control device, respectively; the ultraviolet germicidal lamp being turned on/off according to an air quality condition fed back by the air quality sensing module.

2. The intelligent electric fireplace as claimed in claim 1, wherein the control device controls a light-emitting state of the first light-emitting device to be changed according to the air quality condition fed back by the air quality sensing module.

3. The intelligent electric fireplace as claimed in claim 1, wherein the control device turns on the ultraviolet germicidal lamp for disinfection in a real-time manner according to the air quality condition fed back by the air quality sensing module, after the air quality condition fed back by the air quality sensing module meets a standard, the control device controls the ultraviolet germicidal lamp to stop working.

4. The intelligent electric fireplace as claimed in claim 2, further comprising a switch for manually controlling the ultraviolet germicidal lamp to be turned on/off, wherein when the light-emitting state of the first light-emitting device shows that the air quality condition does not meet a standard, a user manually controls the switch to turn on the ultraviolet germicidal lamp for disinfection.

5. The intelligent electric fireplace as claimed in claim 2, wherein a meshed panel is provided on a front side of the housing, an accommodation space is formed between an inner wall of the housing and the meshed panel, the simulated charcoal and the first light-emitting device are located in the accommodation space; the ultraviolet germicidal lamp is installed in the accommodation space, ambient air is in communication with the accommodation space through the meshed panel, and the ultraviolet germicidal lamp is configured to disinfect the air flowing into the accommodation space.

6. The intelligent electric fireplace as claimed in claim 5, wherein the housing includes a main housing and a middle plate disposed in the main housing, the middle plate divides the main housing into upper and lower spaces, the lower space is defined as the accommodation space, and the ultraviolet germicidal lamp is installed to an underside of the middle plate.

7. The intelligent electric fireplace as claimed in claim 6, wherein the air quality sensing module is far away from the air outlet;
the air inlet is disposed on a rear side of the upper space or above the upper space, the air outlet is disposed on a front side of the upper space, the heating device is disposed in the upper space, a blower is provided in the upper space for forming an air flow from the air inlet to the air outlet; and the air quality sensing module is disposed on a left side or a right side or the rear side of the upper space.

8. The intelligent electric fireplace as claimed in claim 6, wherein the ultraviolet germicidal lamp is arranged horizontally or/and vertically on the underside of the middle plate.

9. The intelligent electric fireplace as claimed in claim 5, further comprising an infrared sensor for detecting whether there is a person in front of the housing, the infrared sensor being connected to the control device; wherein when the infrared sensor detects a person in front of the housing, the control device controls the ultraviolet germicidal lamp to stop working.

10. The intelligent electric fireplace as claimed in claim 6, wherein the middle plate extends obliquely upward from back to front for providing an installation reference surface of the ultraviolet germicidal lamp to extend obliquely upward from back to front, so that the light emitted from the ultraviolet germicidal lamp is irradiated outward toward a front side of the accommodation space.
